(19) United States
(12) Patent Application Publication
(10) Pub. No.: US 2002/0055935 A1
(43) Pub. Date: May 9, 2002

(54) Methods and systems for providing access to one or more databases of information concerning therepeutic and diagnostic agents

(76) Inventor: Michael G. Rosenblum, Sugar Land, TX (US)

Correspondence Address: FULBRIGHT & JAWORSKI L.L.P., SUITE 2400, 600 CONGRESS AVENUE, AUSTIN, TX, 78701, US

(21) Appl. No.: 09/824,523

(22) Filed: Apr. 2, 2001

Related U.S. Application Data
(60) Provisional application No. 60/229,354, filed on Mar. 31, 2000.

(51) Int. Cl. G06F 7/00

(52) U.S. Cl. 707/104.1

(57) ABSTRACT

Methods and systems are provided for multi-level access to one or more databases by information providers and information requesters. The information stored in the one or more databases may include agent profile information and requester profile information. The information may be categorized to form information subsets to facilitate restricted access according to confidentiality levels associated with the subsets, and to facilitate efficient information retrieval by providing searchable access to information categories of interest to different groups.

The present application claims priority to U.S. provisional patent application ser. No. 09/540,829, filed Mar. 31, 2000. The entire text of this disclosure is specifically incorporated by reference herein without disclaimer.

### BACKGROUND OF THE INVENTION

1. Field of the Invention

This invention relates generally to providing searchable access to one or more databases of information concerning therapeutic and diagnostic agents.

2. Description of Related Art

The use of the Internet by individuals and businesses to gain access to information is growing at a phenomenal rate. Internet websites, hosted by manufacturers, retailers, information service providers, and others, provide virtual central locations for providing and accessing all categories of information. Many websites provide access to information databases that contain information of special interest to certain groups of people. Some of these websites are hosted by entities that act as intermediaries between information providers and information requestors. These intermediary websites are specialized as to the subject matter to which information in the database pertains and to the class of individuals and entities that are allowed access to the information.

For example, some intermediaries have hosted websites to facilitate the dissemination of biotechnology related information. Business entities contract with the host to provide information concerning biotechnology products they wish to sell or license. This information is made accessible to entities that are interested in purchasing or obtaining a license to market the products provided the entity signs a confidentiality agreement. The success of these intermediary database websites has been limited for several reasons. They are often not well advertised, so that the number of information providers and information requestors aware of the service provided by the host is too limited. The scope of information encompassed by such databases is often too broad or is poorly organized, making it too difficult to focus a search to find data relevant to a particularized interest. The scope of information provided is sometimes too narrow, thus failing to provide the comprehensive information needed by entities seeking to make well-informed licensing decisions. Also, access to existing biotechnology databases are too restrictive or too broad. Databases that are accessible only to entities that enter into a confidentiality agreement exclude access by others having a bona fide interest in at least some of the information available, whereas databases accessible to the general public fail to contain the more sensitive information that a potential licensee would desire to evaluate a licensure offer.

Moreover, existing intermediary databases do not provide restricted access to different subsets of information in a database to different groups according to the purpose of the group seeking the information. A variety of different groups, as well as the general public, have particularized interests in information concerning therapeutic and diagnostic agents. These groups comprise investors, manufacturers, research entities, charitable foundations, and government agencies. Each group has a different objective with respect to such information, and will therefore have an interest in different categories of information with respect to the agent or its developer. Similarly, the provider of information may associate different levels of sensitivity to different categories of information it provides. Existing databases fail to effectively organize information into multiple categories according to particularized interests of various groups, and fail to provide multiple levels of access according to different levels of sensitivity associated with different categories of information provided.

Therefore, there is a need for an intermediary database system and methods of access thereto, lacking the limitations described above, to facilitate the development and availability of therapeutic and diagnostic agents.

### SUMMARY OF THE INVENTION

The present invention provides systems and methods that overcome existing limitations to enable and further the development and availability of therapeutic and diagnostic agents using computer network technology. The present invention provides a multi-level access approach to providing information concerning therapeutic and diagnostic agents that optimizes the ability of different groups with different interests to search the information while providing optimal security to providers of such information.

According to the present invention a database is provided for storing agent profile information concerning therapeutic and diagnostic agents and for storing requester profile information. The database may be accessible to a plurality of information providers and information requesters from a website by one or more Internet connections. Data entry fields are provided for entering into the database the agent profile and requestor profile information. This information is categorized to form a plurality of information subsets. Criteria are specified for enabling access to each of these information subsets and access to an information subset is enabled for each requester of information that satisfies the specified criteria. A provider of information in the subset may specify the criterion for access to each information subset. The criterion may comprise a confidentiality obligation assumed by the information requester, an ability of a requestor to meet terms for acquisition of rights in an agent, an objective of the requestor with respect to the information sought, or other criterion.

According to the present invention a plurality of providers of information concerning therapeutic and diagnostic agents may access the database electronically and enter agent profile information into the database using data entry fields and point-and-click icons displayed on a video monitor or other suitable device. A plurality of information requesters may also access the database electronically and enter requestor profile information into the database using displayed data entry fields and icons. The data entry fields and icons may be organized to facilitate the categorization of information according to levels of sensitivity associated with the information from the perspective of the agent profile information provider. The fields and icons may also be organized to facilitate the categorization of information according to categories of special interest to different groups of requesters.

The information in the database may be categorized to form searchable subsets wherein the information in a subset is associated with a special interest of one or more of a plurality of groups of information requesters. The information in the database may be categorized to provide information subsets some of which are available to the general public and some of which are restricted to one or more of a plurality of groups comprising investors, marketers, product developers, researchers, and government agencies. The information in the database may also be categorized to provide information subsets comprising subsets of information associated with one of a plurality of different levels of confidentiality, subsets of information descriptive of characteristics of an agent, subsets of information descriptive of intellectual property rights in the agent, subsets of information descriptive of terms for acquisition of rights in the agent, subsets of information descriptive of a market for the agent, as well as other subsets of information for facilitating development and availability of the agent.

Thus, in one respect, the invention is a method for facilitating the development and availability of therapeutic and diagnostic agents. The method includes providing one or more databases for storing agent profile information; providing one or more databases for storing requestor profile information; providing one or more data entry fields for receiving the agent profile information; providing one or more data entry fields for receiving the requester profile information; categorizing information received to form one or more information subsets; determining criteria for access to each information subset; and for each information subset, enabling access to the information subset by each requester that satisfies the criteria for access to the information subset.

In other aspects of the method, the one or more databases for storing agent profile information and the one or more databases for storing requestor profile information may be the same one or more databases. The one or more data entry fields for receiving the agent profile information and the one or more data entry fields for receiving the requestor profile information may be the same one or more data entry fields. The categorizing may include categorizing information received to form multiple information subsets. The criteria for access to at least one of the one or more information subsets may include criteria specified by a provider of information in the at least one information subset. The criteria for access to at least one of the one or more information subsets may include a confidentiality obligation assumed by a requestor, the confidentiality obligation pertaining to information in the at least one information subset. The criteria for access to at least one of the one or more information subsets may include an ability of a requestor to meet terms for acquisition of rights in an agent to which information in the at least one information subset pertains. The criteria for access to at least one of the one or more information subsets may include a research objective of a requester. The criteria for access to at least one of the one or more information subsets may include an investment objective of a requester. The criteria for access to at least one of the one or more information subsets may include a marketing objective of a requester.

In still other aspects of the method, the method may include providing a communications link to a provider of agent profile information in at least one of the one or more information subsets, the link being accessible by a requester of agent profile information if the requester satisfies the criteria for access to the at least one information subset. At least one but less than all of the one or more information subsets may be accessible to all requestors and at least one but less than all of the one or more information subsets may not be accessible to all requesters. The categorizing may include categorizing information to form at least one restricted-access information that is accessible by marketers of therapeutic or diagnostic agents. The categorizing may include categorizing information to form at least one restricted-access information that is accessible by investment entities. The categorizing may include categorizing information to form at least one restricted-access information that is accessible by developers of therapeutic or diagnostic agents.

In other aspects of the method, the categorizing may include categorizing the agent profile information to form multiple information subsets having a first information subset accessible to all requesters, the first information subset having data descriptive of characteristics of an agent; and a second information subset accessible to a pre-determined subgroup of requesters that is less than all requestors, the second information subset having data descriptive of characteristics of the agent. The first information subset may include a classification of an agent and one or more indications for use of the agent. The second information subset may include research data and clinical data pertaining to the agent.

In still other aspects of the method, the categorizing may include categorizing information received to form multiple information subsets including a first information subset that excludes information designated as confidential by any provider of information in the first information subset; and a second information subset including information designated as confidential by a provider of information in the second information subset. The categorizing may include categorizing the agent profile information to form multiple information subsets including an information subset of acquisition information accessible to a pre-determined subgroup of requesters that is less than all requesters. The information subset of acquisition information may include data descriptive of intellectual property rights in an agent. The information subset of acquisition information may include data descriptive of terms for acquiring rights in an agent.

In other aspects of the method, the categorizing may include categorizing the agent profile information to form multiple information subsets including an information subset of marketing information accessible to a pre-determined subgroup of requesters that is less than all requesters. The information subset of marketing information may include data descriptive of a market for an agent. The categorizing may include categorizing the requestor profile information to form multiple information subsets including an information subset of data indicative of a business purpose of a requestor with respect to one or more agents. The requester profile information may include terms for acquisition of rights in an agent offered by a requestor. The categorizing may include categorizing the requestor profile information to form multiple information subsets including an information subset of data indicative of an obligation of confidentiality assumed by a requester with respect to one or more information subsets of data in the one or more databases for storing requestor profile information. The one or more data entry fields for receiving the agent profile information and the one or more data entry fields for receiving the requestor profile information may be displayable and accessible at a website by way of one or more internet connections.

In another respect, the invention is a computer-implemented method for facilitating the development and availability of therapeutic and diagnostic agents. The method includes providing a database for storing agent profile information and for storing requestor profile information; providing data entry fields for receiving the agent profile information and for receiving the requestor profile information; categorizing information received to form multiple information subsets; determining criteria for access to each information subset; and for each information subset, enabling access to the information subset by each requester that satisfies the criteria for access to the information subset.

In still another respect, the invention is a computer network for facilitating the development and availability of therapeutic and diagnostic agents. The computer network includes a database for storing agent profile information and for storing requestor profile information; a set of data entry fields for receiving the agent profile information; a set of data entry fields for receiving the requestor profile information; and an access enabling mechanism configured to enable access to information in one or more information subsets in the database by a requester, wherein access to an information subset is determined from requester profile information indicative of an ability of a requestor to satisfy specified access criteria.

In aspects of the computer network, the computer network may include one or more point-and-click targets for selecting one or more information subsets to be accessed by a requester. The computer network may include one or more point-and-click targets for selecting a subset of data entry fields for entry of information into a selected information subset to be stored in the database. The criteria for access to an information subset may be specifiable by a provider of information in the information subset. Information in the database may be categorized to form the one or more information subsets, the one or more information subsets including one or more information subsets of data descriptive of a developer of therapeutic or diagnostic agents. Information in the database may be categorized to form the one or more information subsets, the one or more information subsets including one or more information subsets of data descriptive of an advertiser of therapeutic or diagnostic agents. Information in the database may also be categorized to form the one or more information subsets, the one or more information subsets including one or more information subsets of data descriptive of a marketer of therapeutic or diagnostic agents. Information in the database may be categorized to form the one or more information subsets, the one or more information subsets including one or more information subsets of data descriptive of an investment entity having an investment objective with respect to one or more therapeutic or diagnostic agents. Information in the database may be categorized to form the one or more information subsets, the one or more information subsets including one or more information subsets of data descriptive of a government agency having an objective with respect to one or more therapeutic or diagnostic agents.

In other aspects of the computer network, the computer network may also include a communications link to a provider of agent profile information, the communications link being accessible by a requester of agent profile information if the requestor satisfies specified criteria. The computer network may include a communications link to one or more inventors of an agent, the communications link being accessible by a requester of access to the one or more inventors if the requester satisfies specified criteria.

In yet another respect, the invention is a multi-level access system for providing shared access to information concerning therapeutic and diagnostic agents between groups. The system includes a database accessible from a website by one more internet connections for storing: agent profile information including data descriptive of a developer of an agent, data descriptive of characteristics of the agent, and data descriptive of terms for acquisition of rights in the agent; and requestor profile information including data descriptive of a requester of information in the database, and data descriptive of terms offered by a requestor for acquisition of rights in an agent. The system also includes a search engine for searching for information in the database; and an access mechanism configured to enable searchable access to subsets of information in the database, the searchable access determined by specified access criteria to enable access to at least one information subset by all requestors and to restrict access to one or more restricted-access information subsets by requesters bound by an obligation of confidentiality with respect to information in the one or more restricted-access subsets.

In aspects of the system, the at least one information subset accessible by all requesters may exclude information designated to be confidential by any provider of information in the at least one information subset; and the one or more restricted-access information subsets may include information designated to be confidential by any provider of information in the one or more restricted-access information subsets. The specified access criteria for the at least one information subset accessible by all requesters may include criteria specified by the provider of information in at least one information subset accessible by all requesters. The specified access criteria for the one or more restricted-access information subsets accessible by requesters bound by an obligation of confidentiality with respect to information in the one or more restricted-access subsets may include criteria specified by the provider of information in at least one of the one or more restricted-access information subsets. The system may also include a communications link to a provider of agent profile information, the communications link being accessible by a requestor of agent profile information if the requestor satisfies the specified access criteria.

Thus, the present invention provides for a multi-level shared access database, wherein the information in the database may be organized into categories of information according to particularized interests of various different groups and provides multiple levels of access according to different levels of sensitivity associated with different categories of information in the database.

These features, as well as other aspects of the present invention, will be more fully understood with reference to the attached figures, and the subsequent discussion of different embodiments and examples of the invention.

### BRIEF DESCRIPTION OF THE DRAWINGS

FIG. 1 is a an illustration of a computer network for implementing an embodiment of the present invention.

### DESCRIPTION OF ILLUSTRATIVE EMBODIMENTS

As a preliminary matter, it should be noted that as used herein, the terms “comprise” (and any form thereof, such as “comprises” and “comprising”), “have” (and any form thereof, such as “has” and “having”), and “include” (and any form thereof, such as “includes” and “including”) are open-ended transitional terms, meaning that a thing or a method that “comprises,” “has,” or “includes” one or more elements possesses those one or more elements, but is not limited to those one or more elements. Thus, and by way of example, a method for facilitating the development and availability of therapeutic and diagnostic agents “comprising” multiple steps has, but is not limited to those steps. That is, the method in questions possesses the recited steps, but does not exclude other steps that are not expressly recited.

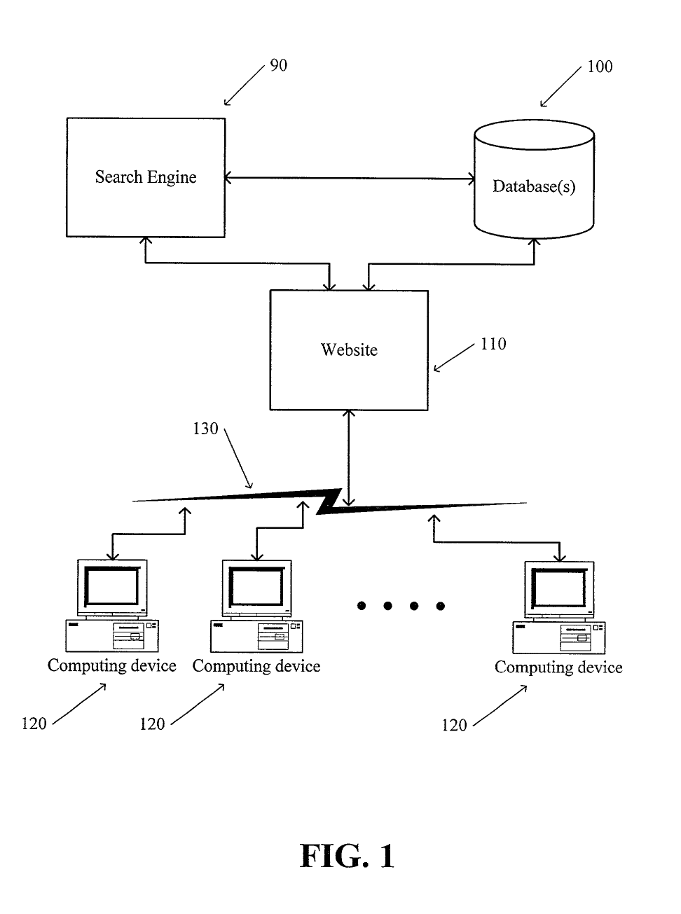

The methods of the present invention may be implemented in a computer network, as shown in FIG. 1, comprising a search engine 90 and a database 100 accessible at a website 110 from a plurality of remote locations 120 by way of connections 130. Multiple databases 100 may be used in this regard, and those databases may or may not be inter-related to each other as is known in the art. Furthermore, the database(s) may be implemented in the form of storage devices such as, for example, magnetic disks, magnetic tapes, optical disks, etc. The connections 130 that provide users access to the information that may be stored in database(s) 100 include any feasible network connection such as, for example, the Internet, an internet, a digital subscriber line (DSL), an intranet, a local area network (LAN), a wide area network (WAN), a T1 connection, a Fibre Channel connection, any combination of these, etc. Website 110 may include one or more pages accessible at remote locations 120 that may be configured to display information on computing devices like personal computers, portable telephones, hand-held devices like a PALM PILOT, or the like, any of which may include a browser. Any of these computing devices may also include an operating system such as a MICROSOFT WINDOWS operating system. In addition, the connections 130 that allow users access to the information in database(s) 100 may be a physical connection, a wireless connection, or a combination of both.

Website 110 may be manifested as a display comprising data entry fields and point-and-click icons that enable access to database(s) 100 for entering into the database(s) agent profile information and requestor profile information. Agent profile information may include information concerning diagnostic or therapeutic agents, as well as information concerning the developer or developers of those agents. Drugs are one example of both therapeutic and diagnostic agents. Thus, therapeutic agents may be drugs or other technology useful in helping to treat living beings suffering from disease or disability of any kind. Therapeutic agents may also be drugs or other technology useful in helping to prevent disease or disability of any kind in living beings. In addition, diagnostic agents may be drugs or other technology useful in helping to diagnose various diseases. Agent profile information providers may enter agent profile information from one or more remote locations 120 via one or more data entry fields. Requestor profile information may include information concerning a requestor of information in database(s) 100. Requestor profile information providers may enter requestor profile information from one or more remote locations 120 via one or more data entry fields. The data entry field or fields for the requester profile information and the data entry field or fields for the agent profile information may be the same data entry field or fields, they may be different, or some of the data entry fields may be the same and others may be different.

Examples of data entry fields for agent profile information include a data entry field for the name of the agent (e.g., product name); a data entry field for the name or names of the inventor of the agent; a data entry field for the organization (such as a company, university, or other organization) responsible for negotiating agreements relating to the agent; a data entry field for the classification of the agent (e.g., drug classification); a data entry field for the classification of the technology with which the agent is related (e.g., technology classification); a data entry field for the structure, or a brief description, of the agent; a data entry field for a proposed mechanism of action of the agent; a data entry field for an indication for use of the agent (i.e., a data entry field calling for the disease categories or applications for which the agent may be useful or applicable); a data entry field for names of agents that compete with or are similar to the relevant agent (i.e., such agents may include those that can compete for the same proposed indications of use); a data entry field for the competitive advantages of the relevant agent over current, or existing, agents; a data entry field for designating the developmental phase of the agent (e.g., the phase of drug development); a data entry field describing the intellectual property coverage associated with the agent (e.g., patents covering the agent or patents pending that cover the agent); a data entry field for the cost associated with purchasing or licensing the agent (e.g., the asking price); and a data entry field for the supporting research data related to the agent (e.g., relevant background information/data relating to the development of the agent). One or more of these data entry fields for receiving agent profile information may be directed to non-confidential information (i.e., information available to any requester). Alternatively, one or more of these data entry fields for receiving agent profile information may be directed to confidential information (i.e., information that is not available to any requester, but that is instead available to requestors that satisfy certain access criteria, discussed below in more detail).

Furthermore, the data entry fields provided for receiving either agent profile information, requester profile information, or both, may have pre-determined descriptions associated with them. By providing such predetermined descriptions, the host of website 110 may further the receipt of agent profile information by diminishing the amount of information that must be typed in by the user, because the user may simply click on a pre-determined description. For example, in the case of a data entry field provided for receiving agent profile information such as the classification of the technology with which the agent is related, such pre-determined descriptions may include one or more of the following: adjuvant, attenuated virus protection, carbohydrates, cell therapy-stem cells/factors, collagen matrix, combinatorial, device, DNA probes, drug delivery-liposomes, drug delivery-oral, drug delivery-other, drug delivery-sustained release, drug delivery-transdermal, gene expression, gene sequencing, generics, immunoassay, immunoglobulin, implantable devices, micropropagation, microspheres, monoclonals, monoclonals-anti-idiotypes, monoclonals-conjugates, monoclonals-humanized abs, monoclonals-transgenic mice, natural product, oligonucleotide ligands, oligonucleotide-antisense/triple helix, oligonucleotide-gene therapy, oligonucleotide-ribozymes, PFOB emulsions, phototherapy, polyclonal antibodies, polyethylene glycol (PEG) products, purines and pyrimidines, rational drug design-computational, rational drug design-peptides, rational drug design-synthetics, recombinant DNA, recombinant protein, resin polymers, screening, separations, service laboratory, synthetics, transcription factors, and transgenics. In yet another embodiment, in the case of a data entry field provided for receiving agent profile information such as supporting research data relating to an agent, such pre-determined descriptions, after which more information may be supplied, may include: background, in vitro studies, animal model data, toxicology studies, histopathology, other relevant pre-clinical data, clinical data, phase I, phase II, and phase III. In addition, text as well as graphics in the form of drawings, schematics, or molecular models may also be provided as agent profile information through, for example, a data entry field for the structure, or brief description, of the agent.

Agent profile information may be stored in one or more databases 100. Requestor profile information may also be stored in one or more databases 100. The one or more databases in which the agent profile information may be stored may be the same one or more databases in which the requestor profile information may be stored, they may be different, or some of the databases may be the same and others may be different. Agent profile information providers and requestor profile information providers may access website 110 using a standard browser such as NETSCAPE NAVIGATOR or MICROSOFT INTERNET EXPLORER. Database(s) 100 and search engine 90 may include software operating on a computer system with associated memory. Configuration of a computer system and implementation of one or more databases, a search engine and a website to accomplish the objectives of the present invention is within the skill of a person of ordinary skill in the art given the disclosure herein. In the interest of clarity, not all features of an actual implementation may be described in this specification. It will be appreciated that in the development of any such actual embodiment, numerous implementation-specific decisions must be made to achieve the developer's specific goals, such as compliance with system-related and business-related constraints, which will likely vary from one implementation to another. Moreover, it will be appreciated that such a development effort might be complex and time-consuming, but would nevertheless be a routine undertaking for those of ordinary skill in the art having the benefit of this disclosure.

Information that is received via the data entry field or fields appearing to a user through remote location 120—which received information may include agent profile information, requestor profile information, or both—may be categorized to form one or more information subsets. Such categorization may be useful in facilitating the development of multiple access levels to the information. Criteria may be determined for allowing access to each information subset, and each requester that satisfies the criteria specified for an information subset may be given access to that information subset. In addition to including the type of information discussed above, requestor profile information may include data, or information, indicative of an ability of a requester to satisfy the specified criteria. The host of website 110 may augment the service provided by the website by conducting an independent investigation of some or all of the representations provided by an information requestor or an information provider.

It will be understood that a provider of agent profile information may also be a requester of agent profile information. Also, an information requestor may request information about other requestors as well as agent profile information. Each provider of agent profile information may specify access criteria for enabling access to the agent profile information it provides, just as each requester information provider may specify access criteria for enabling access to the requester information it provides.

A screening procedure may be employed to determine if an entity desiring to provide agent profile information satisfies certain qualifications determined by the website host to ensure the integrity of agent profile information in database(s) 100. Prospective providers of agent profile information that meet the requisite qualifications may be assigned a password that enables the provider to actually provide, or enter, agent profile information.

The data entry fields and icons that may be used to facilitate the receipt of information may be organized to facilitate the categorization of the information received according to levels of sensitivity associated with the information from the perspective of the information provider. Thus, one or more data entry fields may be provided for entering information pertaining to at least one of one or more categories of agent profile information for each level of access specified for that category of information. Similarly, one or more data entry fields may be provided for entering information pertaining to at least one of one or more categories of requestor profile information for each level of access specified for that category of information. The fields and icons are also preferably organized to facilitate the categorization of information according to categories of special interest to different groups of requesters.

One or more data entry fields and icons may be provided for searching each subset of information in the database(s) 100. For any category of interest to be searched, a data entry field may be provided for entering one or more keywords that may be connected by Boolean or non-Boolean operators. Examples of data entry fields for categories that may be searched to learn about agent profile information include a data entry field for the institution listing information about the agent (i.e., the listing institution); a data entry field for the classification of the agent (e.g., drug classification); a data entry field for other agent classification information (e.g., drug classification other); a data entry field for a mechanism of action of the agent; a data entry field for and indication for use of the agent; a data entry field for the classification of the technology related to the agent; a data entry field for the phase of development of the agent; and a data entry field for text relating to the agent. Like the data entry fields discussed above for providing agent profile information, these data entry fields may contain pre-determined descriptions that may speed the searching process (as opposed to the information entering process discussed above). For example, in the case of a data entry field provided for searching agent profile information such as the classification of the technology with which the agent is related, such pre-determined descriptions may include one or more of the following: adjuvant, attenuated virus production, carbohydrates, cell therapy-stem cells/factors, collagen matrix, combinatorial, device, DNA probes, drug delivery-liposomes, drug delivery-oral, drug delivery-other, drug delivery-sustained release, drug delivery-transdermal, gene expression, gene sequencing, generics, immunoassay, immunoglobulin, implantable devices, micropropagation, microspheres, monoclonals, monoclonals-anti-idiotypes, monoclonals-conjugates, monoclonals-humanized abs, monoclonals-transgenic mice, natural product, oligonucleotide ligands, oligonucleotide-antisense/triple helix, oligonucleotide-gene therapy, oligonucleotide-ribozymes, PFOB emulsions, phototherapy, polyclonal antibodies, polyethylene glycol (PEG) products, purines and pyrimidines, rational drug design-computational, rational drug design-peptides, rational drug design-synthetics, recombinant DNA, recombinant protein, resin polymers, screening, separations, service laboratory, synthetics, transcription factors, and transgenics. In yet another embodiment, in the case of a data entry field provided for searching agent profile information such as the classification of the agent, such pre-determined descriptions may include: any, angoigenisis modulator, apoptosis modulator, antisense, biological agent, biomarker, cell cycle modulator, cell signaling agent, chemotherapeutic agent, cytotoxic agent, co-factor, cytokine, diagnostic agent, DNA, differentiation agent, free radical agent, fusion protein, gene, gene therapy, growth factor, imaging agent, immunomodulator, immunotherapeutic agent, immunotoxin, intermediary metabolism agent, lymphokine, monoclonal antibody, novel analog, p53 modulator, polyamine, polyclonal antibody, protein synthesis inhibitor, receptor modulator, recombinant protein, ribozyme, RNA, targeted therapeutic agent, vaccine, virus, and vitamin. In still another embodiment, in the case of a data entry field provided for searching agent profile information such as the phase of development of an agent, such pre-determined descriptions may include: any, lead, preclinical, IND filed, phase I, phase I/II, phase II, phase II/III, phase III, PLA or NDA filed, FDA action taken, market, and terminated.

Search engine 90 may search the category for text containing the keyword or keywords and the text may be displayed in a scrollable format, emailed to the user, transmitted to the user by phone, facsimile, mail, etc., as is known in the art. In addition, search engine 90 may be configured to alert employees of the website host of the need for a manual search of the information in the database(s) 100. Search engine 90 may also be configured to alert the user to contact the host of the website for more information, etc.

The specified criterion for enabling access to a subset of information associated with a restricted access level may comprise a confidentiality obligation assumed by the information requestor. The requestor may assume the obligation by accessing a confidentiality agreement provided to database(s) 100 by the agent profile information provider. The requestor may enter into the agreement in any of a number of ways. For example, the requester may provide an electronic signature to the agreement. Alternatively, the requestor may download the agreement, sign it and transmit the agreement by facsimile, email, or any other suitable means to the host of website 110. Upon receipt of an executed confidentiality agreement, the host may assign the requestor an access level code. The access level code may determine the information subsets to which the requester may have access. Alternatively, more than one access level code may be assigned to an information requester to designate more than one information subset to which the requester may have access. In such an embodiment, the access level codes assigned to an information requestor may be associated with a single password assigned to the requester that enables access, or allows the requester to gain access, to the relevant information subsets when he or she enters the password into, for example, a data entry field provided therefor.

The specified criterion for access to a subset of information associated with a restricted access level may comprise an ability of a requestor to meet terms for acquisition of rights in an agent to which information in the subset pertains. The terms for acquisition of rights in an agent may be provided to database(s) 100 by way of data entry fields displayed at website 110 by the provider of agent profile information concerning the agent. The requestor may review those terms and indicate whether the requestor can meet, or satisfy, those terms. The requestor may be required to enter data objectively indicative of an ability to meet the terms of acquisition specified by the agent profile information provider.

The specified criterion for access to an information subset associated with a restricted access level may comprise a research objective of the requester. For example, certain subsets of information of particular interest to a researcher of diagnostic or therapeutic agents may comprise certain technical characteristics of an agent considered to be so highly confidential to the information provider that a special access level can be specified to restrict access to groups with a specific research objective.

The specified criterion for access to an information subset associated with a restricted access level may comprise an investment objective of the requestor, enabling an agent profile information provider to restrict access to certain information to groups having one or more specific investment objectives that the information provider prefers. The specified criterion for access to an information subset associated with a restricted access level may also comprise a marketing objective of the requestor, enabling an agent profile information provider to restrict access to certain information to groups having one or more marketing objectives that the information provider prefers.

Criterion may also be specified for accessing a communications link to a provider of information concerning an agent by a requestor of that information. Thus, an information requester that satisfies specified criteria may be provided access via a hyperlink to, for example, an inventor of the agent for the purpose of exchanging additional information concerning the agent.

Certain subsets of information may be formed that contain information that is not considered sensitive to agent profile information providers. These subsets may be accessible to all information requestors accessing database(s) 100. Thus, in a preferred embodiment of the invention, at least one, but less than all, information subsets is accessible to all requesters, and at least one, but less than all, information subsets is not accessible to all requesters. Thus, the information received via one or more of the data entry fields may be categorized to form one or more information subsets that includes an information subset that excludes information designated to be confidential by an information provider, and an information subset that includes information designated to be confidential by an information provider.

The information subsets may include an information subset that is accessible to all requesters, a restricted-access information subset that is accessible to investment entities, a restricted-access information subset that is accessible to marketers of therapeutic or diagnostic agents, a restricted-access subset that is accessible to researchers and/or developers of such agents, a restricted-access subset that is accessible to research entities, as well as a restricted access subset that is accessible to a government or quasi-government agency. Thus, a restricted-access information subset may be accessible to one or more different requestor groups and, at the same time, may not be accessible to requesters that do not fall within one of the groups qualifying for access to the restricted-access subset.

Agent profile information may be categorized to form multiple information subsets, such as multiple agent profile information subsets. One information subset (i.e., a first information subset) may be accessible to all requestors, and may comprise data descriptive of characteristics of one or more agents. Another information subset (i.e., a second information subset) may comprise data descriptive of characteristics of agents, and may be accessible to one or more requestors, but less than all requesters (i.e., a subgroup of requesters), according to the ability of the requester to satisfy criteria specified for access to the restricted data. The first information subset accessible to all requesters may comprise data not associated by a provider of information in the subset with any degree of sensitivity. Such information may include a mechanism of action of an agent and/or one or more indications for use of the agent. The second information subset may include confidential information such as, for example, clinical trial data pertaining to an agent and/or manufacturing processes pertaining to an agent.

The information received may be categorized to form multiple information subsets comprising a subset of acquisition information. This subset may further comprise data descriptive of intellectual property rights in an agent, such as patent rights. This subset may further comprise data descriptive of terms for acquiring rights in an agent, such as royalty percentages, and licensing duration.

The information subsets may comprise a subset of information comprising marketing information, such as data descriptive of a market for an agent, and of similar and competing agents.

The requester profile information may be categorized to form multiple information subsets, such as multiple requestor profile information subsets. Requestor profile information may comprise a subset of information indicative of an ability of the requester to satisfy specified criteria for access to one or more information subsets. Thus, the requestor profile information may further comprise subsets of information indicative of an objective of the requester with respect to agent profile information, such as a marketing, investment, research, or other objective.

Included as the APPENDIX is an example website that may, at the time of filing this disclosure, be found on the World Wide Web at www.advancedmedicines.com, and which demonstrates aspects of the present disclosure.

To summarize, the present invention may be employed to facilitate the development and availability of therapeutic agents. By providing subsets of information focusing on certain categories of information according to levels of confidentiality associated with the data, information providers are provided optimal security, while allowing different levels of access to different groups according to their objectives, and thereby providing each group a more efficient means of searching the database for information of particular interest to the group.

The present invention, therefore, is well adapted to carry out the objects and attains the ends and advantages mentioned herein as well as other ends and advantages made apparent from the disclosure. While exemplary embodiments of the invention have been described, numerous changes and modifications to those embodiments will be readily apparent to those skilled in the art given the disclosure herein, and such changes and modifications are encompassed within the scope of the following claims.

I claim:

1. A method for facilitating the development and availability of therapeutic and diagnostic agents, the method comprising:
providing one or more databases for storing agent profile information;
providing one or more databases for storing requester profile information;
providing one or more data entry fields for receiving the agent profile information;
providing one or more data entry fields for receiving the requestor profile information;
categorizing information received to form one or more information subsets;
determining criteria for access to each information subset; and
for each information subset, enabling access to the information subset by each requestor that satisfies the criteria for access to the information subset.

2. The method of claim 1, wherein the one or more databases for storing agent profile information and the one or more databases for storing requester profile information are the same one or more databases.

3. The method of claim 1, wherein the one or more data entry fields for receiving the agent profile information and the one or more data entry fields for receiving the requestor profile information are the same one or more data entry fields.

4. The method of claim 1, wherein the categorizing comprises categorizing information received to form multiple information subsets.

5. The method of claim 1, wherein the criteria for access to at least one of the one or more information subsets comprises criteria specified by a provider of information in the at least one information subset.

6. The method of claim 1, wherein the criteria for access to at least one of the one or more information subsets comprises a confidentiality obligation assumed by a requestor, the confidentiality obligation pertaining to information in the at least one information subset.

7. The method of claim 1, wherein the criteria for access to at least one of the one or more information subsets comprises an ability of a requester to meet terms for acquisition of rights in an agent to which information in the at least one information subset pertains.

8. The method of claim 1, wherein the criteria for access to at least one of the one or more information subsets comprises a research objective of a requester.

9. The method of claim 1, wherein the criteria for access to at least one of the one or more information subsets comprises an investment objective of a requester.

10. The method of claim 1, wherein the criteria for access to at least one of the one or more information subsets comprises a marketing objective of a requestor.

11. The method of claim 1, further comprising providing a communications link to a provider of agent profile information in at least one of the one or more information subsets, the link being accessible by a requester of agent profile information if the requestor satisfies the criteria for access to the at least one information subset.

12. The method of claim 1, wherein at least one but less than all of the one or more information subsets is accessible to all requesters and at least one but less than all of the one or more information subsets is not accessible to all requesters.

13. The method of claim 1, wherein the categorizing comprises categorizing information to form at least one restricted-access information that is accessible by marketers of therapeutic or diagnostic agents.

14. The method of claim 1, wherein the categorizing comprises categorizing information to form at least one restricted-access information that is accessible by investment entities.

15. The method of claim 1, wherein the categorizing comprises categorizing information to form at least one restricted-access information that is accessible by developers of therapeutic or diagnostic agents.

16. The method of claim 1, wherein the categorizing comprises categorizing the agent profile information to form multiple information subsets comprising:
a first information subset accessible to all requestors, the first information subset having data descriptive of characteristics of an agent; and
a second information subset accessible to a pre-determined subgroup of requestors that is less than all requesters, the second information subset having data descriptive of characteristics of the agent.

17. The method of claim 16, wherein the first information subset comprises a classification of an agent and one or more indications for use of the agent.

18. The method of claim 16, wherein the second information subset comprises research data and clinical data pertaining to the agent.

19. The method of claim 1, wherein the categorizing comprises categorizing information received to form multiple information subsets comprising:
a first information subset that excludes information designated as confidential by any provider of information in the first information subset; and
a second information subset comprising information designated as confidential by a provider of information in the second information subset.

20. The method of claim 1, wherein the categorizing comprises categorizing the agent profile information to form multiple information subsets comprising an information subset of acquisition information accessible to a pre-determined subgroup of requesters that is less than all requestors.

21. The method of claim 20, wherein the information subset of acquisition information comprises data descriptive of intellectual property rights in an agent.

22. The method of claim 20, wherein the information subset of acquisition information comprises data descriptive of terms for acquiring rights in an agent.

23. The method of claim 1, wherein the categorizing comprises categorizing the agent profile information to form multiple information subsets comprising an information subset of marketing information accessible to a pre-determined subgroup of requesters that is less than all requesters.

24. The method of claim 23, wherein the information subset of marketing information comprises data descriptive of a market for an agent.

25. The method of claim 23, wherein the categorizing comprises categorizing the requestor profile information to form multiple information subsets comprising an information subset of data indicative of a business purpose of a requestor with respect to one or more agents.

26. The method of claim 1, wherein the requester profile information comprises terms for acquisition of rights in an agent offered by a requester.

27. The method of claim 1, wherein the categorizing comprises categorizing the requester profile information to form multiple information subsets comprising an information subset of data indicative of an obligation of confidentiality assumed by a requester with respect to one or more information subsets of data in the one or more databases for storing requestor profile information.

28. The method of claim 1, wherein the one or more data entry fields for receiving the agent profile information and the one or more data entry fields for receiving the requestor profile information are displayable and accessible at a website by way of one or more internet connections.

29. A computer-implemented method for facilitating the development and availability of therapeutic and diagnostic agents, the method comprising:
providing a database for storing agent profile information and for storing requester profile information;
providing data entry fields for receiving the agent profile information and for receiving the requestor profile information;
categorizing information received to form multiple information subsets;
determining criteria for access to each information subset; and
for each information subset, enabling access to the information subset by each requestor that satisfies the criteria for access to the information subset.

30. A computer network for facilitating the development and availability of therapeutic and diagnostic agents, comprising;
a database for storing agent profile information and for storing requestor profile information;
a set of data entry fields for receiving the agent profile information;
a set of data entry fields for receiving the requester profile information; and
an access enabling mechanism configured to enable access to information in one or more information subsets in the database by a requestor, wherein access to an information subset is determined from requestor profile information indicative of an ability of a requestor to satisfy specified access criteria.

31. The system of claim 30, further comprising one or more point-and-click targets for selecting one or more information subsets to be accessed by a requestor.

32. The system of claim 30, further comprising one or more point-and-click targets for selecting a subset of data entry fields for entry of information into a selected information subset to be stored in the database.

33. The system of claim 30, wherein criteria for access to an information subset is specifiable by a provider of information in the information subset.

34. The system of claim 30, wherein information in the database is categorized to form the one or more information subsets, the one or more information subsets comprising one or more information subsets of data descriptive of a developer of therapeutic or diagnostic agents.

35. The system of claim 30, wherein information in the database is categorized to form the one or more information subsets, the one or more information subsets comprising one or more information subsets of data descriptive of an advertiser of therapeutic or diagnostic agents.

36. The system of claim 30, wherein information in the database is categorized to form the one or more information subsets, the one or more information subsets comprising one or more information subsets of data descriptive of a marketer of therapeutic or diagnostic agents.

37. The system of claim 30, wherein information in the database is categorized to form the one or more information subsets, the one or more information subsets comprising one or more information subsets of data descriptive of an investment entity having an investment objective with respect to one or more therapeutic or diagnostic agents.

38. The system of claim 30, wherein information in the database is categorized to form the one or more information subsets, the one or more information subsets comprising one or more information subsets of data descriptive of a government agency having an objective with respect to one or more therapeutic or diagnostic agents.

39. The system of claim 30, further comprising a communications link to a provider of agent profile information, the communications link being accessible by a requestor of agent profile information if the requester satisfies specified criteria.

40. The system of claim 30, further comprising a communications link to one or more inventors of an agent, the communications link being accessible by a requestor of access to the one or more inventors if the requestor satisfies specified criteria.

41. A multi-level access system for providing shared access to information concerning therapeutic and diagnostic agents between groups, comprising:
a database accessible from a website by one more internet connections for storing:
agent profile information comprising data descriptive of a developer of an agent, data descriptive of characteristics of the agent, and data descriptive of terms for acquisition of rights in the agent; and
requester profile information comprising data descriptive of a requester of information in the database, and data descriptive of terms offered by a requestor for acquisition of rights in an agent;
a search engine for searching for information in the database; and
an access mechanism configured to enable searchable access to subsets of information in the database, the searchable access determined by specified access criteria to enable access to at least one information subset by all requestors and to restrict access to one or more restricted-access information subsets by requesters bound by an obligation of confidentiality with respect to information in the one or more restricted-access subsets.

42. The system of claim 41, wherein:
the at least one information subset accessible by all requestors excludes information designated to be confidential by any provider of information in the at least one information subset; and
the one or more restricted-access information subsets comprise information designated to be confidential by any provider of information in the one or more restricted-access information subsets.

43. The system of claim 41, wherein the specified access criteria for the at least one information subset accessible by all requesters includes criteria specified by the provider of information in at least one information subset accessible by all requesters.

44. The system of claim 41, wherein the specified access criteria for the one or more restricted-access information subsets accessible by requestors bound by an obligation of confidentiality with respect to information in the one or more restricted-access subsets includes criteria specified by the provider of information in at least one of the one or more restricted-access information subsets.

45. The system of claim 41, further comprising a communications link to a provider of agent profile information, the communications link being accessible by a requester of agent profile information if the requester satisfies the specified access criteria.
